(19) United States
(12) Patent Application Publication
(10) Pub. No.: US 2006/0285315 A1
(43) Pub. Date: Dec. 21, 2006

(54) Hybrid surgical headlight

(75) Inventor: David J. Tufenkjian, Marcellus, NY (US)

Correspondence Address: HISCOCK & BARCLAY, LLP, 2000 HSBC PLAZA, ROCHESTER, NY, 14604-2404, US

(73) Assignee: Welch Allyn, Inc.

(21) Appl. No.: 11/156,990

(22) Filed: Jun. 20, 2005

(51) Int. Cl. F21V 21/084; A61B 1/07

(52) U.S. Cl. 362/105; 362/572

(57) ABSTRACT

The invention comprises, in one form thereof, a surgical headlight having a hybrid design wherein a first low power light source, such as a LED is fixed to a headband such that the surgical headlight is untethered. The surgical headlight further comprises a coupling means that allows for subsequent tethering to a high power light source, such as a fiber-optic light source. The device allows the surgeon to project a spot of light where he needs it, and the ability to plug into a more powerful light source, such as a fiber optic light source, to thereby boost the light to levels that may be required over and above what the low power light source, such as a LED is capable of producing alone.

### FIELD OF THE INVENTION

This invention relates to a surgical headlight, and more particularly, to a surgical headlamp for use on high power and low power lighting applications.

### BACKGROUND OF THE INVENTION

Surgeons and other personnel who work in medical field are often required to illuminate a portion of a patient during surgical procedures. The nature of the surgeon's duties during such procedures requires that they maintain a degree of free use of their hands. These requirements are generally satisfied by illumination assemblies known in the art as surgical headlights. Surgical headlights are mounted on the surgeon's head, and serve to illuminate the patient. The surgical headlight is an optical array held on he surgeon's head with a headband, and having a high intensity light source. Generally, the high intensity light source is connected to the headband by a fiber-optic cable that serves to carry the light from the fiber optic light source to a luminaire. The luminaire is the lighting device attached to the headlight.

Conventional surgical headlights are available in two distinct varieties. The first type of surgical headlight includes a low power light source, such as a LED. This device is typically utilized for surgical applications where less intense directed lighting is required. This type of headlight allows the surgeon free use of both hands. Additionally, the surgeon is untethered and free to move about the surgical area. Unfortunately, the light emitted by such devices is inadequate for many surgical procedures.

Furthermore, the headlight utilized in low power lighting applications may include a rechargeable battery system. During use, the surgeons only indication that the batteries have discharged occurs when the light powered by the batteries begins to dim, or turns off. This event may occur at a critical point of the procedure, requiring the surgeon to stop or seek assistance in replacement of the batteries. Currently, low power headlights that utilize batteries to not include an easily viewable battery status monitor.

The second type of surgical headlight includes a high power light source, such as a fiber-optic light source. These headlights typically remain resident in the operating rooms and are hard wired to a high power light source. While this type of headlight allows the surgeon free use of his hands and a small degree of movement, the headlight remains substantially tethered to the high powered light source, thereby limiting the movement of the surgeon. Additionally, since these headlights typically remain in the operating room, the headlight is usually shared between multiple users and is often found in an undesirable condition, such as covered in sweat. A surgeon entering the operating room generally does not want to use a headlight because it may have just been on the head of the previous user for six hours and thoroughly covered in sweat Unfortunately, because conventional high power headlights remain in the operating room, attached to the light source, this problem is experienced by many surgeons. Furthermore, because high power lighting is not needed during the entire procedure the surgeon is required to either remove the headlight from his head, or remain substantially constrained by the tethered device as noted above.

Conventional headlights that provide either low power or high power lighting are known. One such headlight is disclosed in U.S. Pat. No. 4,516,190 to Kloots. Kloots discloses a surgical headlamp that is removably mounted to a headband and utilizes a remote light source connected via a fiber-optic cable.

U.S. Pat. No. 5,667,291 to Caplan, et al. discloses a small, lightweight, high intensity illumination assembly/or use in dental and medical applications. The illumination assembly includes attachment means for removable attachment to headgear such as eyeglasses, face shields, or headbands, and lenses, loupes. and binoculars associated with such headgear. The illumination assembly is able to achieve extremely light weight by using only a single optical element therein, e.g., an aspheric condensing lens, binary optical element, or holographic optical means, and by piping illumination to the optical element from a remote light source by use of a flexible light guide.

U.S. Pat. No. 6,120,161 to Van Der Bel discloses a video headlight and fiber-optic cable which includes a light and camera assembly adjustably mounted on a headband for assuming a plurality of angular positions relative to the headband. The light and camera assembly includes a light unit closely positioned relative to a video camera unit so that the visual field of the camera unit lies within the lighted field from the light unit in all adjusted positions of the light and camera assembly. The light unit is connected to the forward end of the fiber-optic cable. The rearward end of the fiber-optic cable is connectable to a source of light. The one end of the fiber-optic cable has a flexible, but non-collapsible coupler which bends uniformly when the light and camera assembly is moved relative to the headband.

U.S. Pat. No. 6,224,227 to Klootz discloses an improved surgical headlight assembly having a detachable video camera module. The present invention allows viewers at a remote location to observe an operation procedure on a video monitor from a surgeon's visual perspective. The headlight assembly which is secured via a headband placed around the surgeon's forehead receives light from a light source via a fiber optic cable. The light is sufficient to illuminate the surgeon's area of operation. A video camera is removably affixed to the headlight assembly and, via the use of a roof prism residing within the video camera housing, deflects an erected and accurate image to the video camera, which in turn, transmits the image to a remote video monitor via a coaxial communications cable. A microphone may be provided to allow the surgeon to provide verbal comments to the viewers observing the procedure. The direction of the beam exiting the headlight assembly may be manually adjusted to insure that the beam of light illuminates the area within the focal point of the viewing lens within the video camera, The entire headlight-video camera assembly rests comfortably between the eyes of the surgeon thereby allowing the surgeon to perform the medical procedure in an unhindered manner.

U.S. Patent Application 20040141312 to Henning, et al. discloses a headlamp/camera unit, especially for medical uses comprising at least one lamp, an electronic camera, a support device that supports the at least one lamp and the camera on the head of a person, and an optical sighting mechanism that projects at least one aiming mark into the image field of the camera illuminated by the lamp.

While the devices described above disclose headlights that can be used for either low power or high power lighting, none of the known headlights are capable of being used for both applications. Therefore, a single headlight assembly that can be used for either low power or high power lighting applications is desired.

Further, a single headlight that is capable allowing the user the freedom of motion obtainable by an untethered low power light device, and is easily adaptable to receive a light source to increase the light to levels over and above what the low power light is capable of producing alone is desired.

Further still, a headlight that includes a battery status indicator that is easily monitored by the surgeon during use is desired.

Further still, a headlight that would reduce the need for sharing high power headlights and alleviate associated cleanliness issues is desired.

### SUMMARY OF THE INVENTION

The invention comprises, in one form thereof, a surgical headlight having a hybrid design wherein a first low power light source, such as a LED is fixed to a headband such that the surgical headlight is untethered. The surgical headlight further comprises a coupling means that allows for subsequent tethering to a high power light source, such as a fiber-optic light source. The device allows the surgeon to project a spot of light where he needs it, and the ability to plug into a more powerful light source, such as a fiber optic light source, to thereby boost the light to levels that may be required over and above what the low power light source, such as a LED, is capable of producing alone.

More particularly, the invention includes a surgical headlight comprising a headband having a first light source attached thereto, and a coupling means for attaching said surgical headlight to a second light source.

In another form, the invention includes a method for switching between a low power light source and a high power light source for a surgical headlight. The surgical headlight having the low power light source attached thereto, and a coupling means for attaching said surgical headlight to said high power light source. The method comprises the steps of: illuminating a selected area via said low power light source; discontinuing the illumination of said selected area via said low power light source; attaching said coupling means of said surgical headlight to said high power light source; and illuminating said selected area via said high power light source.

In still another form the invention includes a battery discharge indicator that is positioned on the headlight so that it is within the surgeon's line of sight, thereby providing a means for indicating the state of charge of the batteries, and allowing the user to plan battery changes.

In yet still another form the invention includes a surgical headlight system comprising a headband having a first light source attached thereto; a coupling means for attaching said surgical headlight to a second light source; and at least one battery pack for providing power to said first light source via power line.

An advantage of the present invention is that it allows for a surgeon to utilize a low power light source, such as a LED, and remain untethered from a high power light source, such as a fiber optic light source, until needed.

A further advantage of the present invention is that it allows the surgeon to utilize a single headlight for both high power and low power lighting applications.

An even further advantage of the present invention is that it eliminates the need for sharing a headlight among multiple users.

An even further advantage of the present invention is that it provides a means for monitoring battery status, and for planning the optimal time to replace batteries, thereby minimizing its impact.

### BRIEF DESCRIPTION OF THE DRAWINGS

The above-mentioned and other features and advantages of this invention, and the manner of attaining them, will become apparent and be better understood by reference to the following description of several embodiments of the invention in conjunction with the accompanying drawing, wherein:

FIG. 1 is an illustration of the hybrid surgical headlight of the present invention.

FIG. 2 is an illustration of a particular embodiment of the hybrid surgical headlight of the present invention having a battery discharge indicator.

FIG. 3 is an illustration of a particular embodiment of the hybrid surgical headlight of the present invention having a video camera or a pointer.

Corresponding reference characters indicate corresponding parts throughout the several views. The examples set out herein illustrate several embodiments of the invention but should not be construed as limiting the scope of the invention in any manner.

### DETAILED DESCRIPTION

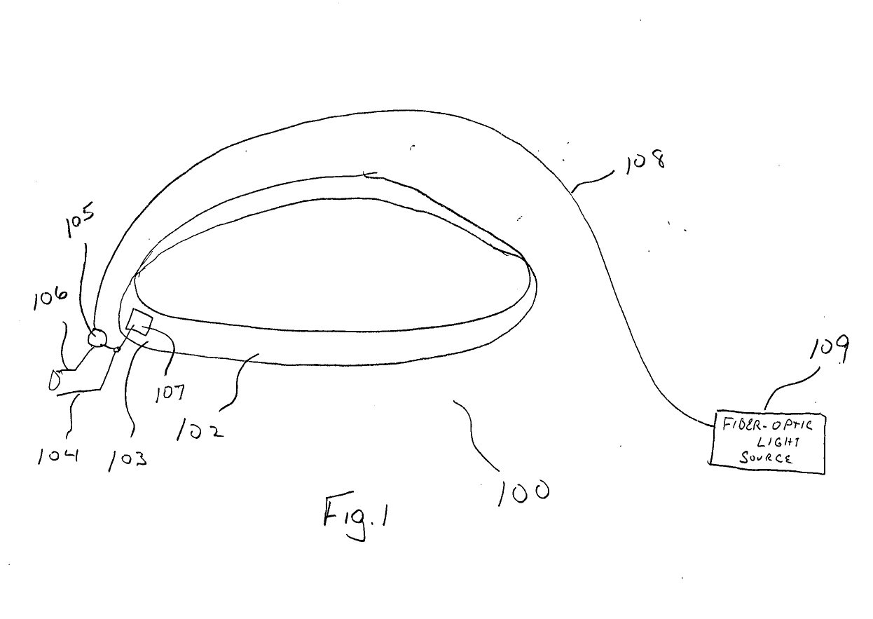

Referring to FIG. 1, there is shown the hybrid surgical headlight 100 of the present invention. The hybrid surgical headlight 100 includes a headband 102, a low power light source means 104, and a coupling means 105 for attaching a high power light source means 106 to the headband 102. High power light sources, such as fiber-optic systems 108, 109 are coupled into the optics and transmit a spot of light. This is achieved by either imaging the iris (not shown), which is the variable hole that adjusts the spot size, or imaging the end of the fiber 108 depending on whether single fiber or multi-fiber are used. Since it is significant that the surgeon be able to place the luminare between his eyes so that the light is co-axial with his vision, the shared optics for the low power lighting means 104, such as a LED, and the high power lighting means 106, such as the fiber, are positioned to provide co-axial illumination. A particular embodiment of the present invention shall include parallel optics, wherein the larger of the two lenses, such as the LED, resides above a smaller lens that images the end of a single fiber. In order to utilize the high power light source, the single fiber is attached to the fiber optic light source. It should be noted that other configurations for positioning the low power and high power light source on a headlight are considered within the scope of the invention.

Referring again to FIG. 1, the headband 102 is an adjustable strap, adapted to be worn about the crown of the surgeon. The headband 102 comprises a front portion 103 proximate to and above the eyes of the surgeon. The front portion 103 includes a means for retaining a low power light source. In the illustrated embodiment 100, the means for retaining the low power light source is a bracket 107, fixedly attached to the headband 102, however other known means for attaching and retaining a low power lighting means 104 to the headband 102 of a surgical headlight 100 are considered within the scope of the invention.

The hybrid surgical headlight 100 of the present invention further comprises a coupling means 105 for selectively receiving and retaining a high power light source means 106. In the illustrated embodiment of the present invention 100, the high power light source means comprises a fiber-optic system 108, 109, and more particularly a fiber-optic line 108 attached to a fiber optic light source 109.

In operation, the surgeon wearing the surgical headlight 100 may use the low powered lighting means 104 that is fixed to the headlight for appropriated applications, wherein the surgeon may enjoy the freedom to move about the operating table, untethered to any light source or device. When an application calls for a high powered lighting means 106, the surgeon attaches the high powered light source, such as a fiber-optic light source 109 and fiber-optic line 108, to the coupling means 105 residing on the headlight 100. After the high powered lighting means 106 is no longer needed, the surgeon easily detaches the high power lighting means 106, or fiber optic line 108, from the coupling means 105 of the surgical headlight 100, wherein he is once again free to move about the operating table and operating room.

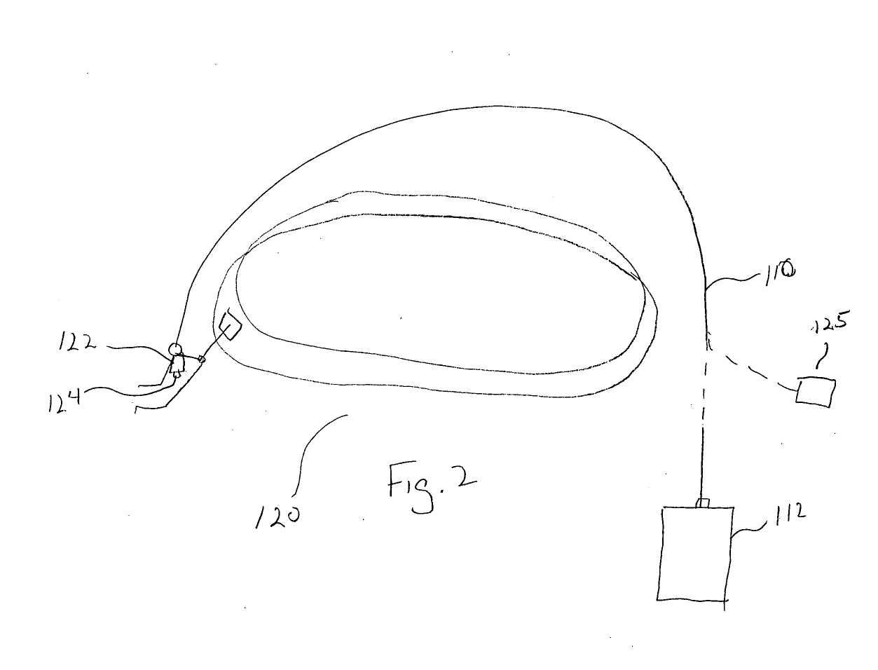

Referring now to FIG. 2, an additional embodiment 120 of the present invention is shown. The embodiment 120 includes a battery discharge indicator 122 attached to the surgical headlight 120. The battery discharge indicator 122 of the present invent invention monitors the state of charge of the batteries 112 being used to power the low powered light means 104, and provides a visual indication of the state of charge of the batteries to the user. Generally, the indicator will be situated on the headlight 120 between the eyes of the surgeon so that he may monitor the state of charge without looking away from his primary viewing area. In a particular embodiment of the present invention, the indication will be in the form of a blinking light, such as a LED 124, or a series of lights (not shown) that provide the user a visual indication of the state of charge. Additionally, the lights may be more than one color, such as green, yellow, and red. By knowing the state of charge of the battery the operator may better plan for the changing of batteries so that he is not required to do so at a critical time of the procedure. An embodiment is also contemplated having a means for connecting to a wall power receptacle 125 attached to power line 110 thereby eliminating the need to replace batteries.

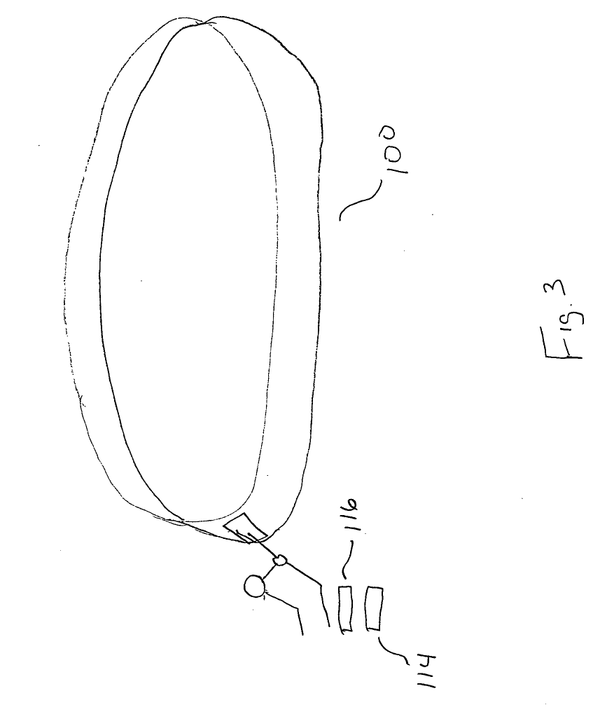

Referring now to FIG. 3., additional embodiments of the present invention are contemplated wherein a video camera 116 or a pointer 114 can be attached to the surgical headlight 100. In operation the video camera 116 attaches to the surgical headlight 100 proximate to the low power lighting means 104, providing a means for filming the area that is illuminated by the surgeon. Additionally, a pointer, such as a laser pointer 104 is attached in a similar manner to the video camera, and can be turned on or off either by the surgeon or by an attendant. The pointer can be utilized to direct attention to landmarks as desired. Additionally, the pointer can be used for documentation or preparing a record of surgery. This embodiment is particularly useful when the attending or the teaching surgeon's hands are occupied and he is attempting to point out a specific landmark.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed for carrying out this invention, but that the invention will include all embodiments falling within the scope and spirit of the appended claims.

I claim:

1. A surgical headlight comprising:
a headband having a first lighting means attached thereto; and
a coupling means for attaching said headlight to a second lighting means.

2. The surgical headlight of claim 1 wherein said first lighting means is a low power light source.

3. The surgical headlight of claim 1 wherein said first lighting means is a LED.

4. The surgical headlamp of claim 1 wherein said second lighting means is high power light source.

5. The surgical headlamp of claim 1 wherein said second lighting means is a fiber-optic light source.

6. The surgical headlight of claim 1 further comprising a DC power receptacle for receiving the power required to illuminate said first lighting means.

7. The surgical headlight of claim 1 further comprising an AC power receptacle for receiving the power required to illuminate said first lighting means.

8. The surgical headlight of claim 1 further comprising a video capture means for capturing an image.

9. The surgical headlight of claim 1 further comprising a laser pointer.

10. The surgical headlight of claim 1 further comprising a battery discharge indicator.

11. The surgical headlight of claim 10 wherein said battery discharge indicator is positioned so that it is visible by the surgeon during use of said surgical headlight.

12. A method of switching between an low power lighting means and a high power lighting means for a surgical headlight, said surgical headlight comprising said low power lighting means, and a coupling means for attaching said high power lighting means thereto, said method comprising the steps of:
illuminating a selected area via said low power lighting means;
discontinuing the illumination of said selected area via said low power lighting means;
attaching said coupling means of said surgical headlight to said high power lighting means; and
illuminating said selected area via said high power lighting means.

13. The method of claim 12 wherein said low power lighting means is a LED.

14. The method of claim 12 wherein said high power lighting means is a fiber-optic light source.

15. A surgical headlight system comprising:
a headband having a first lighting means attached thereto, and a coupling means for attaching said surgical headlight to a second lighting means; and
at least one battery pack for providing power to said first light source via a power cord.

16. The surgical headlight system of claim 15 wherein said low power lighting means is a LED.

17. The surgical headlight system of claim 15 wherein said high power lighting means is a fiber-optic light source.

18. The surgical headlight system of claim 15 further comprising a battery discharge indicator.

19. The surgical headlight of claim 18 wherein said battery discharge indicator is positioned so that it is visible by the operator during use of said surgical headlight.
